(19) United States
(12) Patent Application Publication
(10) Pub. No.: US 2026/0294394 A1
(43) Pub. Date: Oct. 1, 2026

(54) SYSTEM FOR DETERMINING A POSITION OF AN INTERVENTIONAL DEVICE IN AN ULTRASOUND IMAGE BASED ON A POSITION OF THE INTERVENTIONAL DEVICE IN AN X-RAY IMAGE

(71) Applicant: GE Precision Healthcare LLC, Waukesha, WI (US)

(72) Inventors: Olivier GERARD, Oslo (NO); Federico VERONESI, San lazzaro di Savena (IT); Regis VAILLANT, Villebon sur Yvette (FR)

(21) Appl. No.: 19/089,836

(22) Filed: Mar. 25, 2025

(51) Int. Cl. A61B 8/00

(52) U.S. Cl. CPC A61B 8/4416; A61B 8/42; A61B 8/461

(57) ABSTRACT

A medical imaging system may receive an x-ray image of a region of interest of a subject including an interventional device provided in the region of interest of the subject during an interventional procedure. The medical imaging system may determine a rough position of the interventional device in the x-ray image. The medical imaging system may receive ultrasound data of the region of interest of the subject including the interventional device provided in the region of interest of the subject. The medical imaging system may determine a refined position of the interventional device in the ultrasound data based on the rough position of the interventional device in the x-ray image. The medical imaging system may display an ultrasound image of the region of interest of the subject and the interventional device based on determining the refined position of the interventional device in the ultrasound data.

### TECHNICAL FIELD

The present disclosure relates, generally, to a medical imaging system for determining a position of an interventional device in an ultrasound image based on a position of the interventional device in an x-ray image. More specifically, the present disclosure relates to a medical imaging system that displays an ultrasound image of a region of interest of a subject and an interventional device that is provided in the region of interest of the subject during an interventional procedure based on determining the position of the interventional device in ultrasound data using a position of the interventional device in an x-ray image.

### BACKGROUND

A catheterization laboratory, or “cath lab,” may refer to an examination room in a hospital or clinical setting that includes various medical imaging systems that are configured to visualize the structures of the heart. For example, the catheterization laboratory may include an ultrasound system that is configured to acquire ultrasound data and generate ultrasound images, may include an x-ray system that is configured to acquire x-ray data and generate x-ray images, or the like. During an interventional procedure, a clinician might navigate an interventional device through a region of interest of a subject. For instance, during a cardiac procedure, a clinician may navigate the interventional device through a heart of a subject in order to ablate tissue, insert a mitral valve clip, close a left atrial appendage, deliver a stent, remove a thrombus, analyze cardiac function, or the like. Ultrasound images of the heart may more clearly and comprehensively visualize the structures of the heart than as compared to x-ray images. However, ultrasound images might visualize the interventional device less clearly and comprehensively than as compared to x-ray images.

### SUMMARY

This summary introduces concepts that are described in more detail in the detailed description. It should not be used to identify essential features of the claimed subject matter, nor to limit the scope of the claimed subject matter.

In an aspect, a medical imaging system may include a memory configured to store instructions; and one or more processors configured to execute the instructions to: receive an x-ray image of a region of interest of a subject including an interventional device provided in the region of interest of the subject during an interventional procedure; determine a rough position of the interventional device in the x-ray image; receive ultrasound data of the region of interest of the subject including the interventional device provided in the region of interest of the subject; determine a refined position of the interventional device in the ultrasound data based on the rough position of the interventional device in the x-ray image; and display an ultrasound image of the region of interest of the subject and the interventional device based on determining the refined position of the interventional device in the ultrasound data.

In another aspect, a method may include receiving an x-ray image of a region of interest of a subject including an interventional device provided in the region of interest of the subject during an interventional procedure; determining a rough position of the interventional device in the x-ray image; receiving ultrasound data of the region of interest of the subject including the interventional device provided in the region of interest of the subject; determining a refined position of the interventional device in the ultrasound data based on the rough position of the interventional device in the x-ray image; and displaying an ultrasound image of the region of interest of the subject and the interventional device based on determining the refined position of the interventional device in the ultrasound data.

In yet another aspect, a non-transitory computer-readable medium may store instructions that, when executed by one or more processors, cause the one or more processors to: receive an x-ray image of a region of interest of a subject including an interventional device provided in the region of interest of the subject during an interventional procedure; determine a rough position of the interventional device in the x-ray image; acquire ultrasound data of the region of interest of the subject including the interventional device provided in the region of interest of the subject; determine a refined position of the interventional device in the ultrasound data based on the rough position of the interventional device in the x-ray image; and display an ultrasound image of the region of interest of the subject and the interventional device based on determining the refined position of the interventional device in the ultrasound data.

### BRIEF DESCRIPTION OF DRAWINGS

FIG. 1A is a diagram of an example system for determining a position of an interventional device in an ultrasound image based on a position of the interventional device in an x-ray image.

FIG. 1B is a diagram of another example system for determining a position of an interventional device in an ultrasound image based on a position of the interventional device in an x-ray image.

FIG. 2 is a diagram of example components of a medical imaging system of FIG. 1A and/or FIG. 1B in the form of an ultrasound system.

FIG. 3 is a diagram of example components of a medical imaging system of FIG. 1A and/or FIG. 1B in the form of an x-ray system.

FIG. 4 is a diagram of example components of a tracking system of FIG. 1B.

FIG. 5 is a flowchart of an example process for determining a position of an interventional device in an ultrasound image based on a position of the interventional device in an x-ray image.

FIG. 6 is a diagram of an example process for determining a position of an interventional device in an ultrasound image based on a position of the interventional device in an x-ray image.

FIGS. 7A and 7B are diagrams of an example process for displaying ultrasound images of a region of interest of a subject and an interventional device that is provided in the region of interest of the subject during an interventional procedure based on determining the position of the interventional device in ultrasound data using a position of the interventional device in an x-ray image.

FIGS. 8A and 8B are diagrams of an example process for displaying ultrasound images of a region of interest of a subject and an interventional device that is provided in the region of interest of the subject during an interventional procedure based on determining the position of the interventional device in ultrasound data using a position of the interventional device in an x-ray image.

### DETAILED DESCRIPTION

A clinician may utilize one or more medical imaging systems of a catheterization laboratory during an interventional procedure to visualize the position and orientation of an interventional device in a region of interest of a subject as the clinician navigates the interventional device through the region of interest of the subject. As addressed above, ultrasound images of the heart may more clearly and comprehensively visualize the structures of the heart than as compared to x-ray images. However, ultrasound images might visualize the interventional device less clearly and comprehensively than as compared to x-ray images.

Some embodiments herein provide a medical imaging system that may receive an x-ray image of a region of interest of a subject including an interventional device provided in the region of interest of the subject during an interventional procedure; determine a rough position of the interventional device in the x-ray image; receive ultrasound data of the region of interest of the subject including the interventional device provided in the region of interest of the subject; determine a refined position of the interventional device in the ultrasound data based on the rough position of the interventional device in the x-ray image; and display an ultrasound image of the region of interest of the subject and the interventional device based on determining the refined position of the interventional device in the ultrasound data.

In this way, the medical imaging system may determine a position of an interventional device in ultrasound data of a region of interest of a subject based on a position of the interventional device in an x-ray image. The medical imaging system may display an ultrasound image of the region of interest of the subject and the interventional device based on determining the position of the interventional device in the ultrasound data, such as by displaying an ultrasound image of a particular view that clearly and comprehensively depicts the interventional device, such as by displaying an ultrasound image including an adjusted image parameter at a level of the interventional device to more clearly delineate the interventional device, such as by displaying an ultrasound image that was generated based on controlling an ultrasound probe to acquire ultrasound data that corresponds to the position of the interventional device, or the like.

In this way, the embodiments herein provide an improvement in the technical field of medical imaging for interventional procedures by utilizing one or more medical imaging systems to clearly and comprehensively visualize an interventional device during an interventional procedure. Further, in this way, the embodiments herein provide an improvement to medical imaging systems by permitting the medical imaging systems to determine the position of an interventional device in acquired ultrasound data by leveraging information from x-ray images that more clearly depict the interventional device. By determining the position of the interventional device in the ultrasound data, the medical imaging system may display an ultrasound image of a particular view that clearly and comprehensively depicts the interventional device, display an ultrasound image including an adjusted image parameter of the interventional device to more clearly delineate the interventional device, display an ultrasound image that was generated based on controlling an ultrasound probe to acquire ultrasound data that corresponds to the position of the interventional device, or the like.

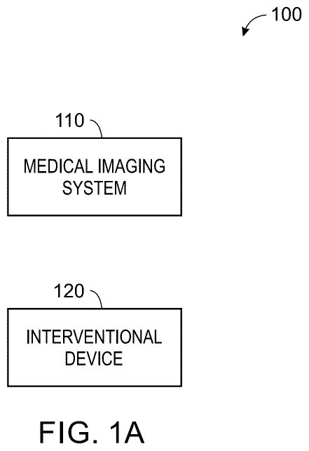

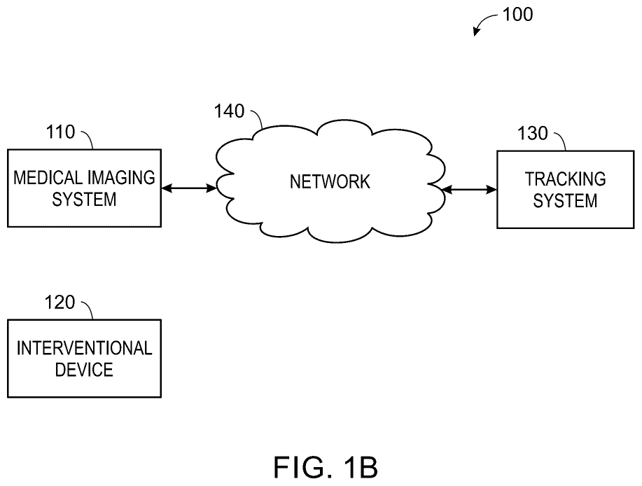

FIG. 1A is a diagram of an example system 100 for determining a position of an interventional device in an ultrasound image based on a position of the interventional device in an x-ray image. As shown in FIG. 1A, the system 100 may include a medical imaging system 110 and an interventional device 120.

The medical imaging system 110 may be configured to receive an x-ray image of a region of interest of a subject including an interventional device provided in the region of interest of the subject during an interventional procedure; determine a rough position of the interventional device in the x-ray image; receive ultrasound data of the region of interest of the subject including the interventional device provided in the region of interest of the subject; determine a refined position of the interventional device in the ultrasound data based on the rough position of the interventional device in the x-ray image; and display an ultrasound image of the region of interest of the subject and the interventional device based on determining the refined position of the interventional device in the ultrasound data. For example, the medical imaging system 110 may be an ultrasound system, an x-ray system, a standalone device, a server, or the like.

The system 100 may include one or more medical imaging systems 110. For example, the system 100 may include a first medical imaging system 110 (e.g., an ultrasound system), a second medical imaging system 110 (e.g., an x-ray system), or the like. Alternatively, the system 100 may include a first medical imaging system 110 (e.g., an ultrasound system), a second medical imaging system 110 (e.g., an x-ray system), a third medical imaging system 110 (e.g., a standalone device), or the like. The one or more medical imaging systems 110 may be configured to collectively, or individually, perform operations described herein.

The interventional device 120 may be any interventional device that can be navigated through a region of interest of a subject. For example, the interventional device 120 may be a catheter, a needle, a trocar, a cannula, or the like. The interventional device 120 may be used for various interventional procedures involving the region of interest. For example, a catheter may be used for delivering a stent to an occluded blood vessel, ablating tissue, analyzing cardiac function, removing a thrombus from an occluded blood vessel, or the like.

The subject may be a person, an animal, a phantom, or the like. The region of interest may be any anatomical region of the subject. For example, the region of interest may be a heart, a brain, an organ, a blood vessel, or the like.

FIG. 1B is a diagram of an example system 100 for determining a position of an interventional device in an ultrasound image based on a position of the interventional device in an x-ray image. As shown in FIG. 1, the system 100 may include a medical imaging system 110, an interventional device 120, a tracking system 130, and a network 140.

The medical imaging system 110 may be configured to receive an x-ray image of a region of interest of a subject including an interventional device provided in the region of interest of the subject during an interventional procedure; determine a rough position of the interventional device in the x-ray image; receive ultrasound data of the region of interest of the subject including the interventional device provided in the region of interest of the subject; determine a refined position of the interventional device in the ultrasound data based on the rough position of the interventional device in the x-ray image; and display an ultrasound image of the region of interest of the subject and the interventional device based on determining the refined position of the interventional device in the ultrasound data. For example, the medical imaging system 110 may be an ultrasound system, an x-ray system, a standalone device, a server, or the like. The system 100 may include one or more medical imaging systems 110.

The interventional device 120 may be any interventional device that can be navigated through a region of interest of a subject. For example, the interventional device 120 may be a catheter, a needle, a trocar, a cannula, or the like. The interventional device 120 may be used for various interventional procedures involving the region of interest. For example, a catheter may be used for delivering a stent to an occluded blood vessel, ablating tissue, analyzing cardiac function, removing a thrombus from an occluded blood vessel, or the like.

The tracking system 130 may be configured to acquire tracking data of the interventional device 120 located within the region of interest of the subject, may be configured to acquire tracking data of the medical imaging system 110, may be configured to acquire tracking data of the x-ray system 120, or the like. For example, the tracking system 130 may be an electromagnetic tracking system, an optical tracking system, an acoustic tracking system, an inertial tracking system, an ultrasound tracking system, or the like.

The network 140 may permit communication between the medical imaging system 110 and the tracking system 130. For example, the network 140 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 are provided as an example. In practice, the system 100 may include additional systems, fewer systems, different systems, or differently arranged systems than those shown in FIGS. 1A and/or 1B. Additionally, or alternatively, a set of systems (e.g., one or more systems) of the system 100 may be integrated into a single system, and/or perform one or more functions described as being performed by another system, or set of systems, of the system 100.

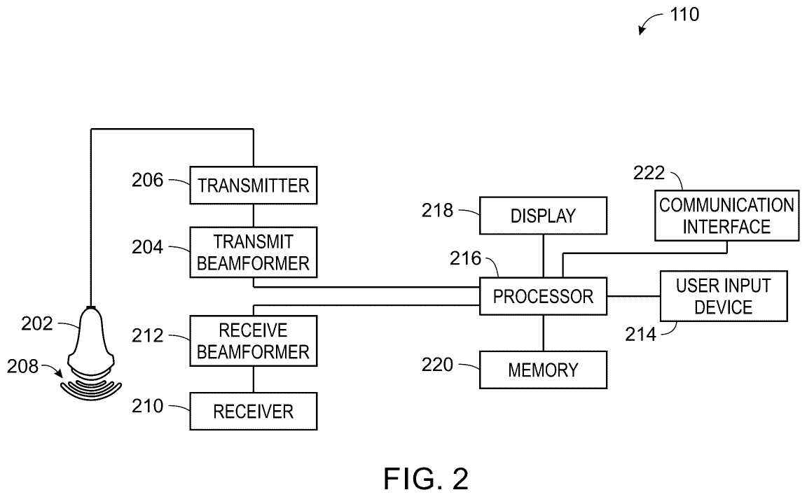

FIG. 2 is a diagram of example components of a medical imaging system of FIG. 1A and/or FIG. 1B in the form of an ultrasound system. As shown in FIG. 2, the medical imaging system 110 may include an ultrasound probe 202, a transmit beamformer 204, a transmitter 206, elements 208 a receiver 210, a receive beamformer 212, a user input device 214, a processor 216, a display 218, a memory 220, and a communication interface 222. The foregoing components may be connected via wired or wireless connections.

In the situation where the medical imaging system 110 is an ultrasound system, the medical imaging system 110 may be configured to acquire ultrasound data of the region of interest of the subject. For example, the medical imaging system 110 may be a two-dimensional (2D) ultrasound system, a three-dimensional (3D) ultrasound system, a four-dimensional (4D) ultrasound system, a Doppler ultrasound system, or the like.

The ultrasound probe 202 may be configured to acquire ultrasound data. For example, the ultrasound probe 202 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d(e 4 D) probe, a low profile wearable patch version of any of the foregoing probes, or the like. According to an embodiment, the ultrasound probe 202 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data. The ultrasound probe 202 may include a lens, an acoustic matching layer, the transducer elements 208, an acoustic dematching layer, and a backing layer.

According to an embodiment, the lens may be configured to direct an ultrasound signal towards the region of interest of the subject. For example, the lens may be silicone, epoxy, rubber, or the like. According to an embodiment, the acoustic matching layer may be configured to facilitate matching of an impedance differential that may exist between the relatively high impedance transducer elements and the relatively low impedance subject. For example, the acoustic matching layer may be graphite, plastic, resin, or the like. According to an embodiment, the transducer elements 208, respectively, may be configured to receive an element specific transmit signal, transform the element specific transmit signal to an ultrasound signal, and transmit the ultrasound signal towards a region of interest. Additionally, or alternatively, the transducer elements 208 may be configured to receive an echo signal reflected by or backscattered from the region of interest, transform the echo signal to an electrical signal, and transmit the electrical signal. For example, the transducer elements 308 may be piezoelectric materials, such as Pb(Mg_{1/3}Nb_{2/3})O₃—PbTiO₃ (“PMN-PT”), Pb(In_{1/2}Nb_{1/2})O₃—Pb(Mg_{1/3}Nb_{2/3})O₃—PbTiO₃ (“PIN-PMN-PT”), Pb(ZrTi) (“PZT”), or the like. According to an embodiment, the acoustic dematching layer may be configured to decrease insertion losses and enhance a frequency bandwidth of the transducer elements 208. For example, the acoustic dematching layer may be tungsten carbide, silicon carbide, or the like. According to an embodiment, the backing layer may be configured to attenuate ultrasound signals directed from the transducer elements 208 in a direction opposite to the subject, and attenuate ultrasound signals deflected by a housing of the ultrasound probe 202. For example, the backing layer may be an epoxy, a metal, or the like.

The transmit beamformer 204 may be configured to apply delay times to electrical signals provided to the elements 208 to focus corresponding ultrasound signals at the region of interest. The transmitter 206 may be configured to transmit electrical signals to the elements 208 to drive the elements 208 to emit ultrasound signals towards the region of interest. The elements 208 may be configured to receive the electrical signals from the transmitter 206, convert the electrical signals into ultrasound signals, and emit the ultrasound signals towards the region of interest. The elements 208 may be configured to receive echo ultrasound signals that are back-scattered by the region of interest, convert the echo ultrasound signals into electrical signals, and provide the electrical signals to the receiver 210. The receiver 210 may be configured to receive electrical signals from the elements 208, and provide the electrical signals to the receive beamformer 212. The receive beamformer 212 may apply delay times to the electrical signals received from the elements 208.

The user input device 214 may be configured to receive a user input, and provide the user input to the processor 216. For example, the user input device 214 may be a user interface, a touch screen display, a keyboard, a keypad, a mouse, a button, a switch, a microphone, or the like. Additionally, or alternatively, the user input device 214 may be configured to sense information. For example, the user input device 214 may sense information from an electro-magnetic positioning system, an inertial measurement system, an accelerometer, a gyroscope, an actuator, or the like.

The processor 216 may be configured to perform the operations as described herein. For example, the processor 216 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 216 may be implemented in hardware, firmware, or a combination of hardware and software. The processor 216 may include one or more processors 216 configured to perform the operations described herein. For example, a single processor 216 may be configured to perform all of the operations described herein. Alternatively, multiple processors 216, collectively, may be configured to perform all of the operations described herein, and each of the multiple processors 216 may be configured to perform a subset of the operations described herein. For example, a first processor 216 may perform a first subset of the operations described herein, a second processor 216 may be configured to perform a second subset of the operations described herein, etc.

The processor 216 may be configured to control the ultrasound probe 202 to acquire ultrasound data. The processor 216 may be configured to control which of the elements 208 are active, and control the shape of a beam emitted from the ultrasound probe 202. The processor 216 may generate ultrasound images for display. For example, the processor 216 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 218 may be configured to display information. For example, the display 218 may be a monitor, an LED display, a cathode ray tube, a projector display, a touchscreen, tablet computer, mobile phone, or the like. The display 218 may display ultrasound images based on the ultrasound data in real-time. For example, the display 218 may display the ultrasound images within one second, two seconds, five seconds, etc., of the ultrasound data being acquired by the ultrasound probe 202.

The memory 220 may be configured to store information and/or instructions for use by the processor 216. The memory 220 may be a non-transitory computer-readable medium. For example, the memory 220 may be a random access memory (RAM), a read only memory (ROM), and/or another type of dynamic or static storage device (e.g., a flash memory, a magnetic memory, and/or an optical memory) that stores information and/or instructions for use by the processor 216. The memory 220 may be configured to store instructions that, when executed by the processor 216, cause the processor 216 to perform the operations described herein.

The communication interface 222 may be configured to enable the processor 216 to communicate with other systems, such as via a wired connection, a wireless connection, or a combination of wired and wireless connections. For example, the communication interface 222 may include an Ethernet interface, an optical interface, a coaxial interface, an infrared interface, a radio frequency (RF) interface, a universal serial bus (USB) interface, a Wi-Fi interface, a cellular network interface, or the like.

The number and arrangement of the components of the ultrasound system 110 shown in FIG. 2 are provided as an example. In practice, the medical imaging system 110 may include additional components, fewer components, different components, or differently arranged components than those shown in FIG. 2. Additionally, or alternatively, a set of components (e.g., one or more components) of the medical imaging system 110 may perform one or more functions described as being performed by another set of components of the medical imaging system 110

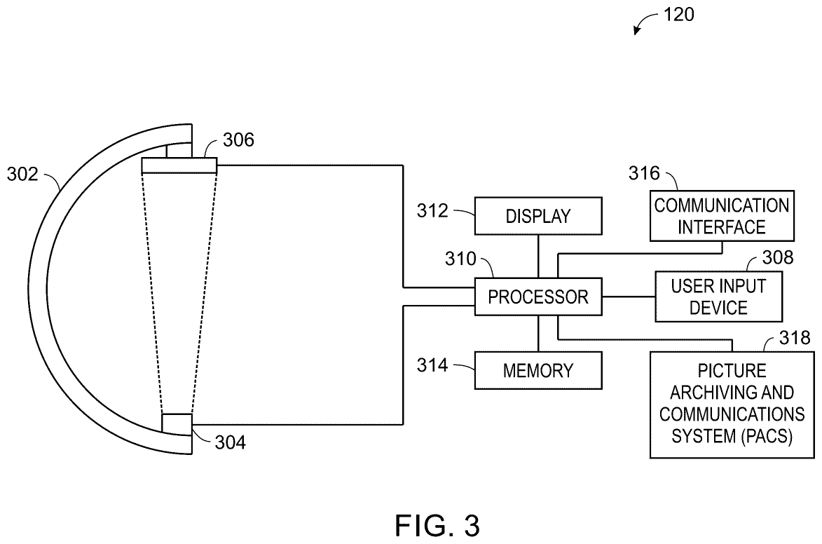

FIG. 3 is a diagram of example components of a medical imaging system of FIG. 1A and/or FIG. 1B in the form an x-ray system. As shown in FIG. 3, the x-ray system 120 may include a c-arm 302, an x-ray source 304, an x-ray detector 306, a user input device 308, a processor 310, a display 312, a memory 314, a communication interface 316, and a picture archiving and communications system (PACS) 318.

In the situation where the medical imaging system 110 is an x-ray system, the medical imaging system 110 may be configured to acquire x-ray data of the region of interest of the subject. For example, the medical imaging system 110 may be a direct radiography system, a computed radiography system, a computed tomography system, or the like

The c-arm 302 may be configured to support the x-ray source 304 and the x-ray detector 306. The x-ray source 304 may be configured to emit X-ray radiation in the form of an X-ray beam towards the subject and the x-ray detector 306. The x-ray detector 306 may be configured to detect X-ray radiation emitted by the X-ray source 304 and attenuated by the subject.

The user input device 308 may be configured to receive a user input, and provide the user input to the processor 310. For example, the user input device 308 may be a user interface, a touch screen display, a keyboard, a keypad, a mouse, a button, a switch, a microphone, or the like. Additionally, or alternatively, the user input device 308 may be configured to sense information. For example, the user input device 308 may sense information from an electro-magnetic positioning system, an inertial measurement system, an accelerometer, a gyroscope, an actuator, or the like.

The processor 310 may be configured to control the x-ray source 304 and the x-ray detector 306. For example, the processor 310 may be a CPU, a GPU, an APU, a microprocessor, a microcontroller, a DSP, an FPGA, an ASIC, or the like. The processor 310 may be implemented in hardware, firmware, or a combination of hardware and software. The processor 310 may include one or more processors 310 configured to perform the operations described herein. For example, a single processor 310 may be configured to perform all of the operations described herein. Alternatively, multiple processors 310, collectively, may be configured to perform all of the operations described herein, and each of the multiple processors 310 may be configured to perform a subset of the operations described herein. For example, a first processor 310 may perform a first subset of the operations described herein, a second processor 310 may be configured to perform a second subset of the operations described herein, etc.

The display 312 may be configured to display information. For example, the display 312 may be a monitor, an LED display, a cathode ray tube, a projector display, a touchscreen, tablet computer, mobile phone, or the like. The memory 314 may be configured to store information and/or instructions for use by the processor 310. The memory 314 may be a non-transitory computer-readable medium. For example, the memory 314 may be a RAM, a ROM, a flash memory, a magnetic memory, an optical memory, or the like. The memory 314 may be configured to store instructions that, when executed by the processor 310, cause the processor 310 to perform the operations described herein.

The communication interface 316 may be configured to enable the processor 310 to communicate with other systems, such as via a wired connection, a wireless connection, or a combination of wired and wireless connections. For example, the communication interface 316 may include an Ethernet interface, an optical interface, a coaxial interface, an infrared interface, an RF interface, a USB interface, a Wi-Fi interface, a cellular network interface, or the like. The PACS 318 may be configured to communicate with external systems and/or networks to permit users at various locations to access x-ray images.

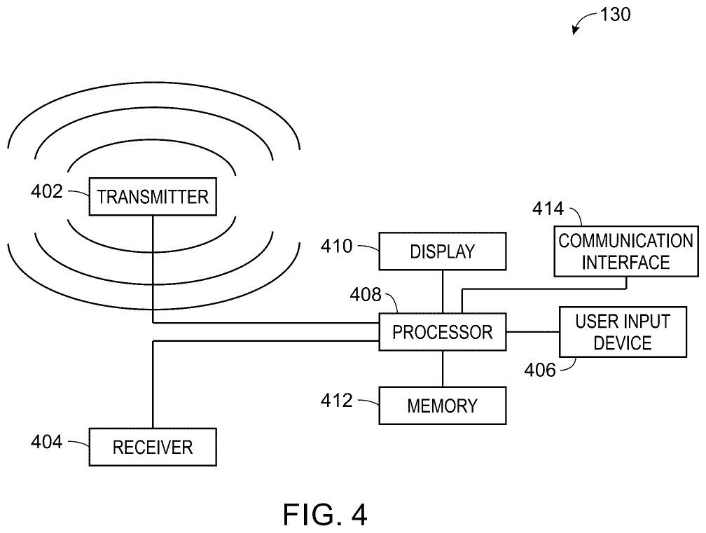

FIG. 4 is a diagram of example components of a tracking system 130 of FIG. 1B. As shown in FIG. 4, the tracking system 130 may include a transmitter 402, a receiver 404, a user input device 406, a processor 408, a display 410, a memory 412, and a communication interface 414.

The transmitter 402 may be configured to generate a magnetic field. The receiver 404 may be configured to output a signal in response to the magnetic field generated by the transmitter 402. The processor 408 may receive the output signal from the receiver 404, and acquire tracking data that identifies a position and/or an orientation of the receiver 404. According to an embodiment, the receiver 404 may be attached to the medical imaging system 110, the interventional device 120, or the like. For example, the receiver 404 may be attached to the ultrasound probe 202 to track a position and/or an orientation of the ultrasound probe 202. As another example, the receiver 404 may be attached to the x-ray source 304 or the x-ray detector 306 to track a position and/or an orientation of the x-ray source 304 or the x-ray detector 306. As another example, the receiver 404 may be attached to the interventional device 120 to track a position and/or an orientation of the interventional device 120. As another example, the receiver 404 may be attached to the feature in the region of interest.

The user input device 406 may be configured to receive a user input, and provide the user input to the processor 408. For example, the user input device 406 may be a touch screen display, a keyboard, a keypad, a mouse, a button, a switch, a microphone, or the like. Additionally, or alternatively, the user input device 406 may be configured to sense information. For example, the user input device 406 may sense information from an electro-magnetic positioning system, an inertial measurement system, an accelerometer, a gyroscope, an actuator, or the like.

The processor 408 may be configured to perform the operations as described herein. For example, the processor 408 may be a CPU, a GPU, an APU, a microprocessor, a microcontroller, a DSP, an FPGA, an ASIC, or the like. The processor 408 may be implemented in hardware, firmware, or a combination of hardware and software. The processor 408 may include one or more processors 408 configured to perform the operations described herein. For example, a single processor 408 may be configured to perform all of the operations described herein. Alternatively, multiple processors 408, collectively, may be configured to perform all of the operations described herein, and each of the multiple processors 408 may be configured to perform a subset of the operations described herein. For example, a first processor 408 may perform a first subset of the operations described herein, a second processor 408 may be configured to perform a second subset of the operations described herein, etc.

The processor 408 may be configured to control the transmitter 402 to acquire tracking data. The processor 408 may be configured to control excitations of the transmitter 402 to generate a magnetic field. The processor 408 may acquire tracking data based on controlling the transmitter 402.

The display 410 may be configured to display information. For example, the display 410 may be a monitor, an LED display, a cathode ray tube, a projector display, a touchscreen, tablet computer, mobile phone, or the like. The display 410 may display the tracking data in real-time. For example, the display 410 may display the tracking data within one second, two seconds, five seconds, etc., of the tracking data being acquired.

The memory 412 may be configured to store information and/or instructions for use by the processor 408. The memory 412 may be a non-transitory computer-readable medium. For example, the memory 412 may be a RAM, a ROM, a flash memory, a magnetic memory, an optical memory, or the like. The memory 412 may be configured to store instructions that, when executed by the processor 408, cause the processor 408 to perform the operations described herein.

The communication interface 414 may be configured to enable the processor 408 to communicate with other systems, such as via a wired connection, a wireless connection, or a combination of wired and wireless connections. For example, the communication interface 414 may include an Ethernet interface, an optical interface, a coaxial interface, an infrared interface, an RF interface, a USB interface, a Wi-Fi interface, a cellular network interface, or the like.

The number and arrangement of the components of the tracking system 130 shown in FIG. 4 are provided as an example. In practice, the tracking system 130 may include additional components, fewer components, different components, or differently arranged components than those shown in FIG. 4. Additionally, or alternatively, a set of components (e.g., one or more components) of the tracking system 130 may perform one or more functions described as being performed by another set of components of the tracking system 130.

Although FIG. 4 depicts the tracking system 130 as being an electromagnetic tracking system, it should be understood that the embodiments herein are applicable to other types of tracking systems, such as optical tracking systems, acoustic tracking systems, ultrasound tracking systems, AI-based tracking methods, or the like

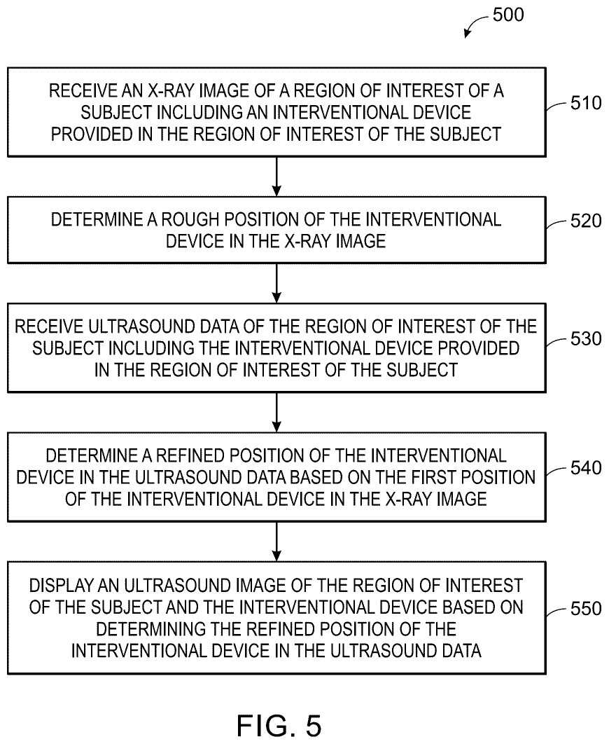

FIG. 5 is a flowchart of an example process 500 for determining a position of an interventional device in an ultrasound image based on a position of the interventional device in an x-ray image. The process 500 may be performed by the medical imaging system 110. Further, the process 500 may be performed by one or more medical imaging systems 110. For example, a first medical imaging system 110 (e.g., an ultrasound system) may perform one or more operations of the process 500, a second medical imaging system 110 (e.g., an x-ray system) may perform one or more operations of the process 500, etc.

As shown in FIG. 5, the process 500 may include receiving an x-ray image of a region of interest of a subject including an interventional device provided in the region of interest of the subject during an interventional procedure (operation 510). For example, the medical imaging system 110 may receive an x-ray image of a region of interest of a subject including the interventional device 120 provided in the region of interest of the subject during an interventional procedure. The medical imaging system 110 may receive the x-ray image during an interventional procedure in which the interventional device 120 is navigated through the region of interest of the subject. During the interventional procedure, the interventional device 120 may be positioned within and navigated through the region of interest. Accordingly, the x-ray image may depict the interventional device 120.

As further shown in FIG. 5, the process 500 may include determining a rough position of the interventional device in the x-ray image (operation 520). For example, the medical imaging system 110 may determine a rough position of the interventional device 120 in the x-ray image.

According to an embodiment, the medical imaging system 110 may determine the rough position of the interventional device 120 in the x-ray image using an image processing technique. For example, the image processing technique may be a segmentation technique, a pattern matching technique, a feature extraction technique, an image analysis technique, an edge detection technique, an image registration technique, or the like. The medical imaging system 110 may analyze the x-ray image using the image processing technique, and determine the rough position of the interventional device 120 in the x-ray image.

According to another embodiment, the medical imaging system 110 may determine the rough position of the interventional device 120 in the x-ray image using an AI model. For example, the medical imaging system 110 may input the x-ray image into the AI model, and determine the rough position of the interventional device 120 in the x-ray image based on an output of the AI model. The AI model may be a convolutional neural network (CNN) model, a residual neural network, a random forest model, a decision tree model, an artificial neural network (ANN), a Naïve Bayes model, a decision tree, a recurrent neural network (RNN), a logistic regression model, a support vector machine, or the like.

The medical imaging system 110 may determine one or more coordinates corresponding to the rough position. For example, the one or more coordinates may be coordinates of a coordinate system of the medical imaging system 110 and the tracking system 130. The coordinate system may be a coordinate system of a catheterization laboratory including the medical imaging system 110 and the x-ray system 120. According to an embodiment, the one or more coordinates may be coordinates of a shape of the interventional device 120 in the x-ray image. Additionally, or alternatively, the one or more coordinates may be coordinates of a search space corresponding to the shape of the interventional device 120 in the x-ray image. For example, the search space may be a three-dimensional (3D) shape that is based on the shape of the interventional device 120 in the x-ray image. For instance, the shape of the interventional device 120 in the x-ray image may be a two-dimensional (2D) shape because the x-ray image is 2D. Because the x-ray image is 2D, the 3D position and orientation of the interventional device 120 in the region of interest might not be entirely ascertainable from the x-ray image. Accordingly, the search space might represent a delineation of potential positions and/or orientations of the interventional device 120 in the region of interest.

According to an embodiment, the medical imaging system 110 may determine the search space based on the one or more coordinates of the rough position. For example, the medical imaging system 110 may extend the one or more coordinates in one or more directions to determine the search space. Additionally, or alternatively, the medical imaging system 110 may determine the search space based on the one or more coordinates of the rough position and a predetermined model. For example, the predetermined model may correspond to the search space, and may be a 3D model. The medical imaging system 110 may determine the coordinates of the search space based on the one or more coordinates of the rough position and the dimensions of the 3D model. The 3D model may be a similar shape as the shape of the interventional device 120. Alternatively, the 3D model may be a different shape as the shape of the interventional device 120. In any event, the search space may encompass the shape of the interventional device 120 and may potentially encompass additional space around the interventional device 120 to account for potential positions and orientations of the interventional device 120.

As further shown in FIG. 5, the process 500 may include receiving ultrasound data of the region of interest of the subject including the interventional device provided in the region of interest of the subject (operation 530). For example, the medical imaging system 110 may receive ultrasound data of the region of interest of the subject including the interventional device 120 provided in the region of interest of the subject. The medical imaging system 110 may receive the ultrasound data during the interventional procedure based on a user manipulating the ultrasound probe 202 relative to the subject.

As further shown in FIG. 5, the process 500 may include determining a refined position of the interventional device in the ultrasound data based on the rough position of the interventional device in the x-ray image (operation 540). For example, the medical imaging system 110 may determine a refined position of the interventional device 120 in the ultrasound data based on the rough position of the interventional device 120 in the x-ray image.

According to an embodiment, the refined position may be one or more coordinates of the interventional device 120 in the ultrasound data that are determined based on the one or more coordinates of the rough position. The one or more coordinates may be coordinates of a coordinate system of the medical imaging system 110 and the tracking system 130. The coordinate system may be a coordinate system of a catheterization laboratory including the medical imaging system 110 and the tracking system 130.

According to an embodiment, the refined position may be one or more coordinates of the interventional device 120 in the ultrasound data that are determined based on coordinates of the x-ray image. Alternatively, the refined position may be one or more coordinates of the interventional device 120 in the ultrasound data that are determined based on a coordinate system of the tracking system 130. Alternatively, the refined position may be one or more coordinates of the interventional device 120 in the ultrasound data that are determined based on a coordinate system of a medical imaging system 110 that acquired the x-ray image.

According to an embodiment, the medical imaging system 110 may determine the refined position based on the one or more coordinates of the rough position. For example, if the one or more coordinates of the rough position delineate the search space, the medical imaging system 110 may analyze the ultrasound data in the search space to determine the refined position of the interventional device 120 in the search space. In other words, the search space might represent a delineation of potential positions and/or orientations of the interventional device 120 in the region of interest, and the refined position of the interventional device 120 in the ultrasound data might represent the actual position and/or orientation of the interventional device 120 in the region of interest.

According to an embodiment, the medical imaging system 110 may determine the refined position based on a position of the ultrasound probe 202 in the x-ray image. For example, the medical imaging system 110 may analyze the x-ray image to determine a position of the ultrasound probe 202 in the x-ray image, and determine the refined position of the interventional device 120 in the ultrasound data based on the position of the ultrasound probe 202 in the x-ray image.

According to an embodiment, the medical imaging system 110 may determine the refined position of the interventional device 120 in the ultrasound data using an image processing technique. For example, the image processing technique may be a segmentation technique, a pattern matching technique, a feature extraction technique, an image analysis technique, an edge detection technique, an image registration technique, or the like. The medical imaging system 110 may analyze the ultrasound data using the image processing technique, and determine the refined position of the interventional device 120 in the ultrasound data.

According to another embodiment, the medical imaging system 110 may determine the refined position of the interventional device 120 in the x-ray image using an AI model. For example, the medical imaging system 110 may input the ultrasound data into the AI model, and determine the refined position of the interventional device 120 in the ultrasound data based on an output of the AI model. The AI model may be a CNN model, a residual neural network, a random forest model, a decision tree model, an ANN, a Naïve Bayes model, a decision tree, an RNN, a logistic regression model, a support vector machine, or the like.

According to an embodiment, the medical imaging system 110 may analyze the search space in the ultrasound data to determine the refined position of the interventional device 120 in the ultrasound data. As described above, the search space might delineate the potential position and/or orientation of the interventional device 120 in the ultrasound data. By analyzing only the search space instead of the entire dataset of the ultrasound data, the medical imaging system 110 may more quickly and efficiently determine the refined position of the interventional device 120 in the ultrasound data.

As further shown in FIG. 5, the process 500 may include displaying an ultrasound image of the region of interest of the subject and the interventional device based on determining the refined position of the interventional device in the ultrasound data (operation 550). For example, the medical imaging system 110 may display an ultrasound image of the region of interest of the subject and the interventional device 120 in the region of interest of the subject based on determining the refined position of the interventional device 120 in the ultrasound data.

According to an embodiment, the medical imaging system 110 may display the ultrasound image based on determining a view that depicts the interventional device 120 in the region of interest of the subject. For example, the medical imaging system 110 may determine a view that depicts the interventional device 120 in the region of interest of the subject, and display an ultrasound image corresponds to the determined view. As examples, the medical imaging system 110 may display one or more ultrasound images corresponding to various axes of the interventional device 120 in order to assist the user in assessing the positioning of the interventional device 120 in the region of interest of the subject.

According to an embodiment, the medical imaging system 110 may display the ultrasound image based on adjusting an image parameter of the ultrasound data at a level of the interventional device 120. For example, the image parameter may be a color, an opacity, a hue, a brightness, a pattern, or the like. The medical imaging system 110 may adjust the image parameter of the ultrasound data at a level of the interventional device 120 to improve the visibility of the interventional device 120 in the ultrasound image and/or to enhance the interventional device 120 in the ultrasound image. According to an embodiment, the medical imaging system 110 may adjust an image parameter of a particular portion of the interventional device 120, such as the tip of a catheter, a tip of a guidewire, the “wings” of a mitral valve clip, or the like.

According to an embodiment, the medical imaging system 110 may display the ultrasound image based on controlling the ultrasound probe 202 to acquire ultrasound data corresponding to the refined position of the interventional device 120. For example, the medical imaging system 110 may determine the refined position of the interventional device 120 in the ultrasound data, and control the ultrasound probe 202 to acquire additional ultrasound data that corresponds to the refined position.

Although FIG. 5 depicts particular operations and a particular sequence of operations, it should be understood that other embodiments may include different operations and/or a different sequence of operations.

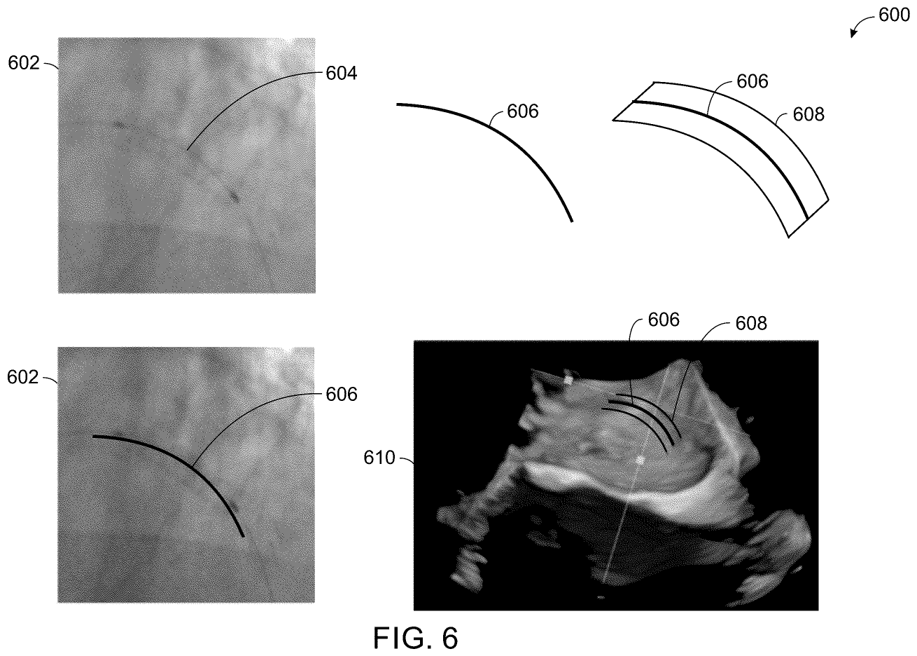

FIG. 6 is a diagram of an example process 600 for determining a position of an interventional device in an ultrasound image based on a position of the interventional device in an x-ray image. As shown in FIG. 6, the medical imaging system 110 may receive an x-ray image 602 of a region of interest of a subject including an interventional device 120 provided in the region of interest of the subject. As further shown in FIG. 6, the medical imaging system 110 may determine a rough position 604 of the interventional device 120 in the x-ray image 602. Further, as shown, the medical imaging system 110 may determine a shape 606 of the interventional device 120 in the x-ray image 602. The medical imaging system 110 may determine a search space 608 based on the shape 606. The search space might represent a delineation of potential positions and/or orientations of the interventional device 120 in the region of interest. As further shown in FIG. 6, the medical imaging system 110 may receive ultrasound data 610 of the region of interest of the subject including the interventional device 120 provided in the region of interest of the subject. As further shown in FIG. 6, the medical imaging system 110 may determine a refined position of the interventional device 120 provided in the ultrasound data based on the rough position of the interventional device 120 in the x-ray image 602. For example, as shown, the medical imaging system 110 may determine a refined position of the interventional device 120 in the ultrasound data 610 based on the search space 608 and/or the shape 606.

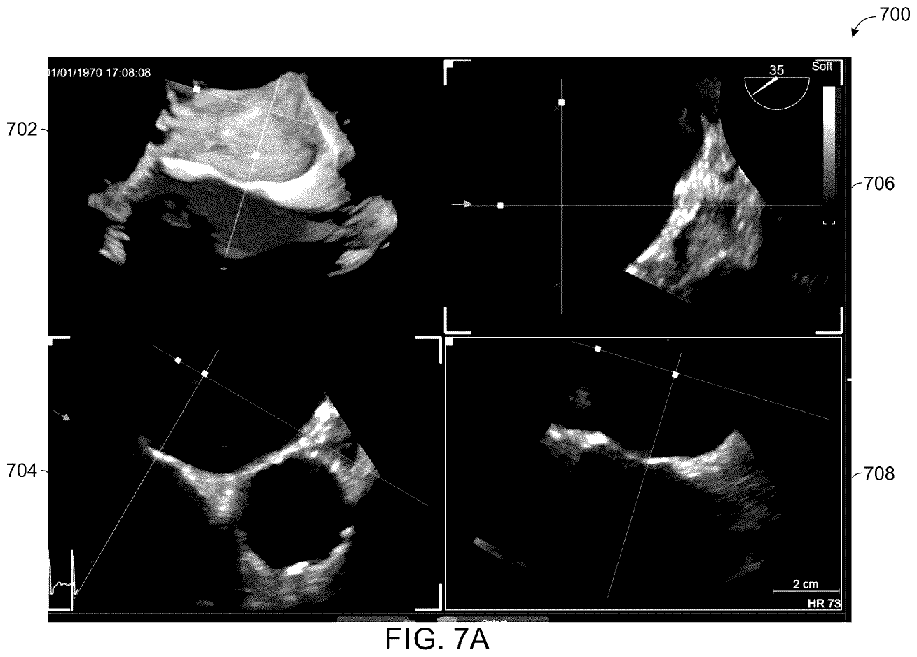

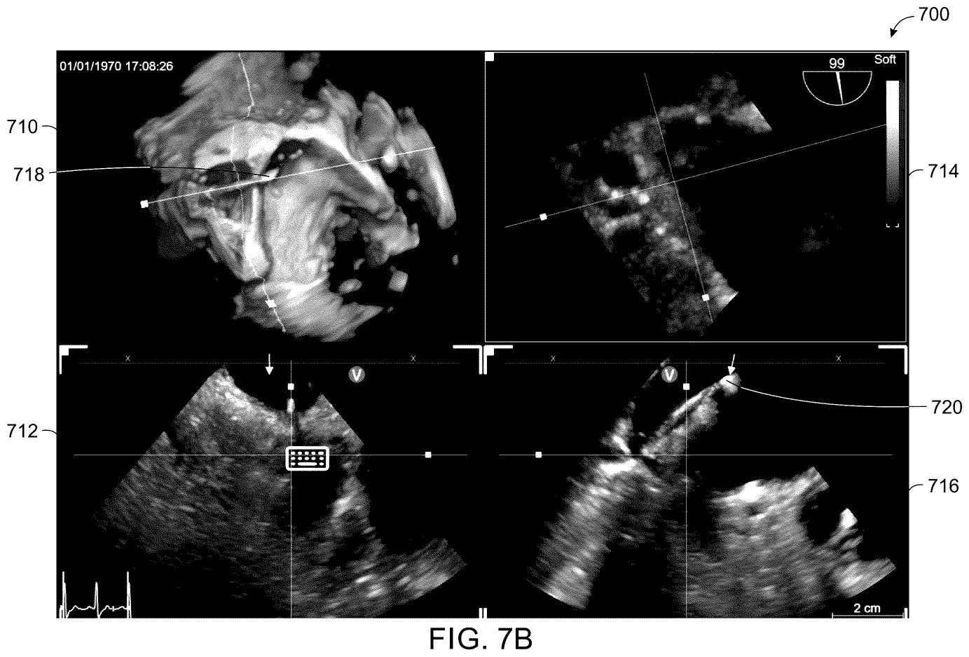

FIGS. 7A and 7B are diagrams of an example process 700 for displaying ultrasound images of a region of interest of a subject and an interventional device that is provided in the region of interest of the subject during an interventional procedure based on determining the position of the interventional device in ultrasound data using a position of the interventional device in an x-ray image. As shown in FIG. 7A, the medical imaging system 110 may display a 3D ultrasound image 702 corresponding to a region of interest of a subject, may display a first 2D ultrasound image 704 corresponding to a first view of the region of interest of the subject, may display a second 2D ultrasound image 706 corresponding to a second view of the region of interest of the subject, and may display a third 2D ultrasound image 708 corresponding to a third view of the region of interest of the subject. An interventional device 120 (e.g., a catheter) may be provided in the region of interest of the subject. However, the interventional device 120 might not be readily visible in the 3D ultrasound image 702, the first 2D ultrasound image 704, the second 2D ultrasound image 706, and/or the third 2D ultrasound image 708. As shown in FIG. 7B, the medical imaging system 110 may display a 3D ultrasound image 710 corresponding to a region of interest of a subject, may display a first 2D ultrasound image 712 corresponding to a first view of the region of interest of the subject, may display a second 2D ultrasound image 714 corresponding to a second view of the region of interest of the subject, and may display a third 2D ultrasound image 716 corresponding to a third view of the region of interest of the subject. As shown by reference numbers 718 and 720, the interventional device 120 may be visible in the 3D ultrasound image 710 and the third 2D ultrasound image 716. The medical imaging system 110 may update the display of the ultrasound images from the display of FIG. 7A to the display of FIG. 7B based on determining the refined position of the interventional device 120 in the ultrasound data, in a similar manner as described above in connection with FIG. 5.

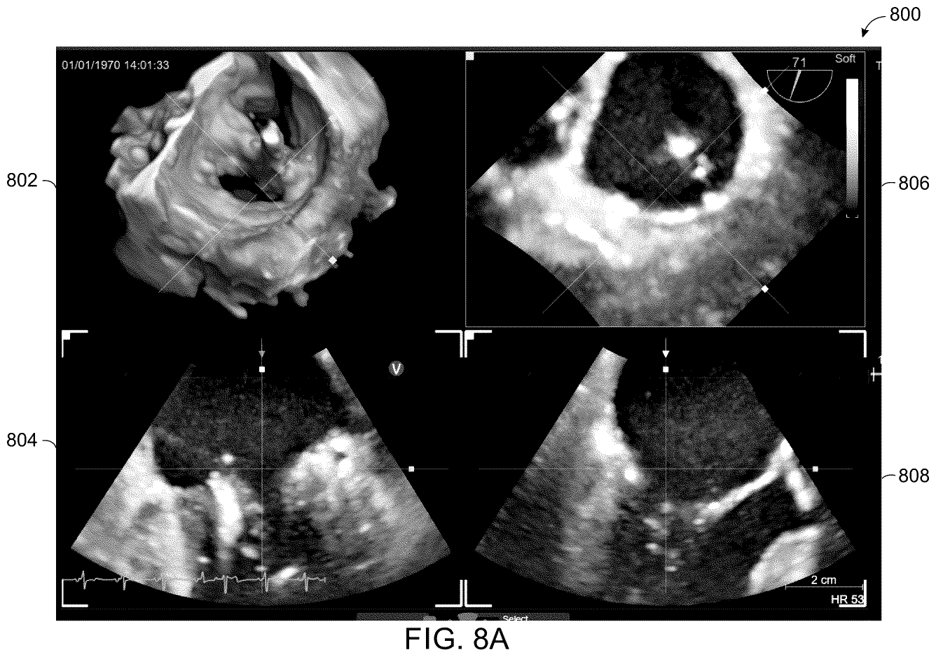

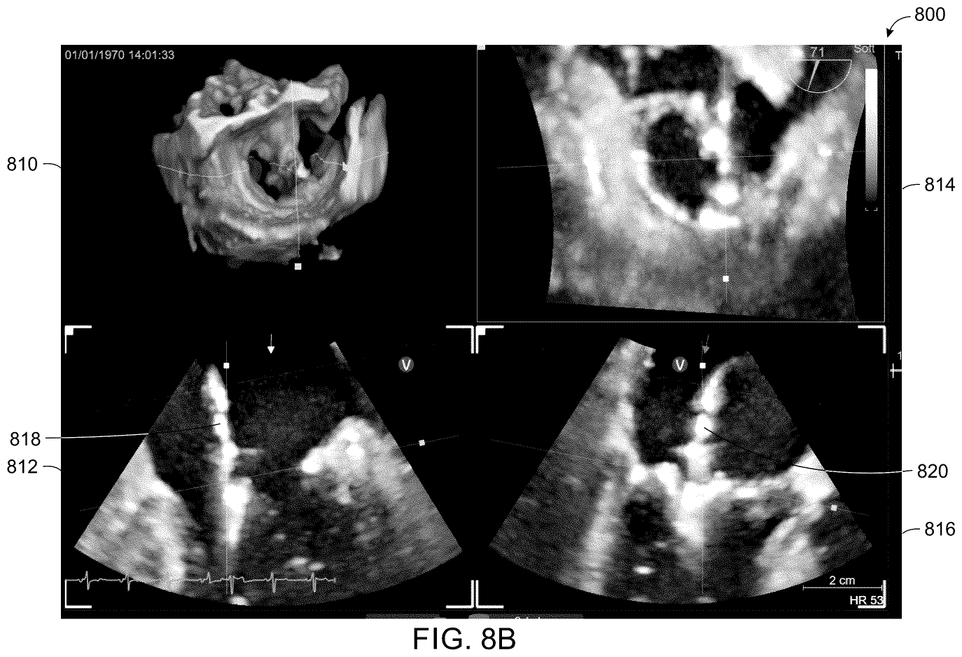

FIGS. 8A and 8B are diagrams of an example process 800 for displaying ultrasound images of a region of interest of a subject and an interventional device that is provided in the region of interest of the subject during an interventional procedure based on determining the position of the interventional device in ultrasound data using a position of the interventional device in an x-ray image. As shown in FIG. 8A, the medical imaging system 110 may display a 3D ultrasound image 802 corresponding to a region of interest of a subject, may display a first 2D ultrasound image 804 corresponding to a first view of the region of interest of the subject, may display a second 2D ultrasound image 806 corresponding to a second view of the region of interest of the subject, and may display a third 2D ultrasound image 808 corresponding to a third view of the region of interest of the subject. An interventional device 120 (e.g., a mitral valve clip) may be provided in the region of interest of the subject. However, the interventional device 120 might not be readily visible in the 3D ultrasound image 802, the first 2D ultrasound image 804, the second 2D ultrasound image 806, and/or the third 2D ultrasound image 808. As shown in FIG. 8B, the medical imaging system 110 may display a 3D ultrasound image 810 corresponding to a region of interest of a subject, may display a first 2D ultrasound image 812 corresponding to a first view of the region of interest of the subject, may display a second 2D ultrasound image 814 corresponding to a second view of the region of interest of the subject, and may display a third 2D ultrasound image 816 corresponding to a third view of the region of interest of the subject. As shown by reference numbers 818 and 820, the interventional device 120 may be visible in the first 2D ultrasound image 812 and the third 2D ultrasound image 816. The medical imaging system 110 may update the display of the ultrasound images from the display of FIG. 8A to the display of FIG. 8B based on determining the refined position of the interventional device 120 in the ultrasound data, in a similar manner as described above in connection with FIG. 5.

According to an embodiment, the medical imaging system 110 may use an AI model. The one or more AI models may be associated with a training phase, a deployment phase, and a monitoring phase. In the training phase, the medical imaging system 110 may receive and process training data to generate a trained model. The training data may be generated, received, or otherwise obtained from internal and/or external resources.

Generally, the trained model may include a set of variables (e.g., nodes, neurons, filters, or the like) that are tuned (e.g., weighted, biased, or the like) to different values via the application of the training data. According to an embodiment, the training process may employ supervised, unsupervised, semi-supervised, and/or reinforcement learning processes to train the model. According to an embodiment, a portion of the training data may be withheld during training and/or used to validate the trained model.

For supervised learning processes, the training data may include labels or scores that may facilitate the training process by providing a ground truth. For example, the labels or scores may indicate an output of the model. Training may proceed by feeding a training dataset including the training data into the model. The model may have variables set at initialized values (e.g., at random, based on Gaussian noise, based on pre-trained values, or the like). The model may generate an output based on the training dataset being input to the model. The output may be compared with the corresponding label or score (e.g., the ground truth) indicating the known output, which may then be back-propagated through the model to adjust the values of the variables. This process may be repeated for a plurality of samples at least until a determined loss or error is below a predefined threshold. According to an embodiment, some of the training data may be withheld and used to further validate or test the trained model.

For unsupervised learning processes, the training data may not include pre-assigned labels or scores to aid the learning process. Instead, unsupervised learning processes may include clustering, classification, or the like, to identify naturally occurring patterns in the training data. As an example, the training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data with pre-assigned labels or scores and training data without pre-assigned labels or scores may be used to train the model.

When reinforcement learning is employed, an agent (e.g., an algorithm) may be trained to make a decision from the training data through trial and error. For example, based on making a decision, the agent may then receive feedback (e.g., a positive reward if the prediction was above a predetermined threshold), adjust its next decision to maximize the reward, and repeat until a loss function is optimized.

After being trained, the trained model may be stored and subsequently applied by the medical imaging system 110 during the deployment phase. For example, during the deployment phase, the trained model executed by the medical imaging system 110 may receive input data. During the deployment phase, the trained model may perform one or more operations as described in connection with FIG. 5.

The embodiments herein provide an improvement in the technical field of medical imaging for interventional procedures by utilizing an x-ray system and an ultrasound system to clearly and comprehensively visualize an interventional device during an interventional procedure. Further, in this way, the embodiments herein provide an improvement to ultrasound systems by permitting the ultrasound systems to determine the position of an interventional device in acquired ultrasound data by leveraging information from x-ray images that more clearly depict the interventional device. By determining the position of the interventional device in the ultrasound data, the ultrasound system may display an ultrasound image of a particular view that clearly and comprehensively depicts the interventional device, display an ultrasound image including an adjusted image parameter of the interventional device to more clearly delineate the interventional device, display an ultrasound image that was generated based on controlling an ultrasound probe to acquire ultrasound data that corresponds to the position of the interventional device, or the like.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A medical imaging system comprising:
a memory configured to store instructions; and
one or more processors configured to execute the instructions to:
receive an x-ray image of a region of interest of a subject including an interventional device provided in the region of interest of the subject during an interventional procedure;
determine a rough position of the interventional device in the x-ray image;
receive ultrasound data of the region of interest of the subject including the interventional device provided in the region of interest of the subject;
determine a refined position of the interventional device in the ultrasound data based on the rough position of the interventional device in the x-ray image; and
display an ultrasound image of the region of interest of the subject and the interventional device based on determining the refined position of the interventional device in the ultrasound data.

2. The medical imaging system of claim 1, wherein the one or more processors are further configured to:
determine a view that depicts the interventional device in the ultrasound data based on determining the refined position of the interventional device in the ultrasound data; and
display the ultrasound image corresponding to the view.

3. The medical imaging system of claim 1, wherein the one or more processors are further configured to:
adjust an image parameter of the ultrasound data at a level of the interventional device; and
display the ultrasound image based on adjusting the image parameter.

4. The medical imaging system of claim 1, wherein the one or more processors are further configured to:
control an ultrasound probe to acquire additional ultrasound data corresponding to the refined position of the interventional device; and
display the ultrasound image based on controlling the ultrasound probe to acquire the additional ultrasound data.

5. The medical imaging system of claim 1, wherein the one or more processors are further configured to:
determine a search space based on the rough position of the interventional device in the x-ray image; and
determine the refined position of the interventional device in the ultrasound data based on the search space.

6. The medical imaging system of claim 1, wherein the one or more processors are further configured to:
determine the refined position of the interventional device in the ultrasound data based on tracking data acquired by a tracking system.

7. The medical imaging system of claim 1, wherein the one or more processors are further configured to:
determine the refined position of the interventional device in the ultrasound data based on a position of an ultrasound probe in the x-ray image.

8. A method comprising:
receiving an x-ray image of a region of interest of a subject including an interventional device provided in the region of interest of the subject during an interventional procedure;
determining a rough position of the interventional device in the x-ray image;
acquiring ultrasound data of the region of interest of the subject including the interventional device provided in the region of interest of the subject;
determining a refined position of the interventional device in the ultrasound data based on the rough position of the interventional device in the x-ray image; and
displaying an ultrasound image of the region of interest of the subject and the interventional device based on determining the refined position of the interventional device in the ultrasound data.

9. The method of claim 8, further comprising:
determining a view that depicts the interventional device in the ultrasound data based on determining the refined position of the interventional device in the ultrasound data; and
displaying the ultrasound image corresponding to the view.

10. The method of claim 8, further comprising:
adjusting an image parameter of the ultrasound data at a level of the interventional device; and
displaying the ultrasound image based on adjusting the image parameter.

11. The method of claim 8, further comprising:
controlling an ultrasound probe to acquire additional ultrasound data corresponding to the refined position of the interventional device; and
displaying the ultrasound image based on controlling the ultrasound probe to acquire the additional ultrasound data.

12. The method of claim 8, further comprising:
determining a search space based on the rough position of the interventional device in the x-ray image; and
determining the refined position of the interventional device in the ultrasound data based on the search space.

13. The method of claim 8, further comprising:
determining the refined position of the interventional device in the ultrasound data based on tracking data acquired by a tracking system.

14. The method of claim 8, further comprising:
determining the refined position of the interventional device in the ultrasound data based on a position of an ultrasound probe in the x-ray image.

15. A non-transitory computer-readable medium storing instructions that, when executed by one or more processors, cause the one or more processors to:
receive an x-ray image of a region of interest of a subject including an interventional device provided in the region of interest of the subject during an interventional procedure;
determine a rough position of the interventional device in the x-ray image;
receive ultrasound data of the region of interest of the subject including the interventional device provided in the region of interest of the subject;
determine a refined position of the interventional device in the ultrasound data based on the rough position of the interventional device in the x-ray image; and
display an ultrasound image of the region of interest of the subject and the interventional device based on determining the refined position of the interventional device in the ultrasound data.

16. The non-transitory computer-readable medium of claim 15, wherein the instructions further cause the one or more processors to:
determine a view that depicts the interventional device in the ultrasound data based on determining the refined position of the interventional device in the ultrasound data; and
display the ultrasound image corresponding to the view.

17. The non-transitory computer-readable medium of claim 15, wherein the instructions further cause the one or more processors to:
adjust an image parameter of the ultrasound data at a level of the interventional device; and
display the ultrasound image based on adjusting the image parameter.

18. The non-transitory computer-readable medium of claim 15, wherein the instructions further cause the one or more processors to:
control an ultrasound probe to acquire additional ultrasound data corresponding to the refined position of the interventional device; and
display the ultrasound image based on controlling the ultrasound probe to acquire the additional ultrasound data.

19. The non-transitory computer-readable medium of claim 15, wherein the instructions further cause the one or more processors to:
determine a search space based on the rough position of the interventional device in the x-ray image; and
determine the refined position of the interventional device in the ultrasound data based on the search space.

20. The non-transitory computer-readable medium of claim 15, wherein the instructions further cause the one or more processors to:
determine the refined position of the interventional device in the ultrasound data based on tracking data acquired by a tracking system or determine the refined position of the interventional device in the ultrasound data based on a position of an ultrasound probe in the x-ray image.
